(19) United States
(12) Patent Application Publication
(10) Pub. No.: US 2009/0005326 A1
(43) Pub. Date: Jan. 1, 2009

(54) SINGLE DOSE ROXITHROMYCIN

(75) Inventors: Yerramilli V.S.N. Murthy, Apex, NC (US); Edward Obare, Raleigh, NC (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: IDEXX Laboratories, Inc., Westbrook, ME (US)

(21) Appl. No.: 12/143,270

(22) Filed: Jun. 20, 2008

Related U.S. Application Data
(60) Provisional application No. 60/929,401, filed on Jun. 26, 2007.

(51) Int. Cl. A61K 31/7048; A61P 31/04

(52) U.S. Cl. 514/29

(57) ABSTRACT

The invention relates to a method of treating a bacterial infection in an animal by administering a single dose of roxithromycin.

### CROSS-REFERENCE TO RELATED APPLICATIONS

This application claims the benefit of U.S. provisional application Ser. No. 60/929,401, filed Jun. 26, 2007, the contents of which are expressly incorporated herein by reference.

### FIELD OF THE INVENTION

The invention is directed to a method of treating an infection in an animal by administering a single dose of roxithromycin.

### BACKGROUND OF THE INVENTION

Roxithromycin is a semi-synthetic macrolide antibiotic derived from erythromycin. Roxithromycin includes the same 14-membered lactone ring as erythromycin, however, the ketone carbonyl group in erythromycin is replaced with an N-oxime side chain. The structure of roxithromycin is:

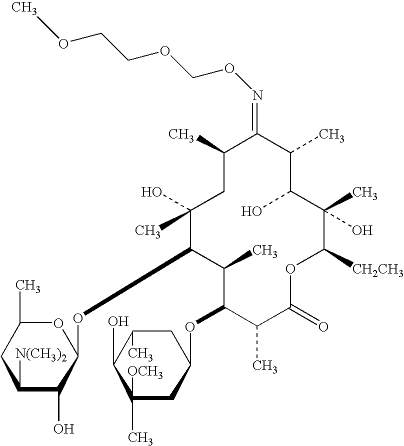

Roxithromycin is sold under the trade names Surlid, Rulide, Biaxsig, Roxar, and Roximycin and is commercially available as a tablet or oral suspension. Roxithromycin has a similar antimicrobial spectrum as erythromycin but is more effective against certain gram-negative bacteria, particularly Legionella pneumophlia. The mechanism of action of the macrolide antibiotics is thought to involve inhibition of bacterial protein synthesis by binding reversibly to subunit 50S of the bacterial ribosome, thereby inhibiting translocation of peptidyl-tRNA.

Roxithromycin is indicated for the treatment of mild to moderate infections of the ear, nose and throat, respiratory tract, skin and skin structure, and genito-urinary tract caused by susceptible strains of organisms in humans. The treatment regimen typically involves administering 1 or 2 tablets per day for 5 to 10 days. The half-life of roxithromycin in humans is about 10-12 hours. Roxithromycin is not indicated for administration to animals other than humans.

U.S. Pat. No. 6,987,093 discloses a method for treating a respiratory infection in a human with a single dose of azithromycin.

E. Lavy et al., J. Vet. Pharmacol. Therap., 18, 382-384 (1985) discloses orally administering roxithromycin to dogs.

A continual problem with antibiotic therapy is the emergence of resistant microbial strains. A method of treating microbial infections having a reduced risk of developing treatment-resistant strains is therefore desirable. It is believed that a method of treating an infection with a single dose of roxithromycin reduces the risk of the emergence of microbial strains that are resistant to roxithromycin compared to methods that involve multiple doses of roxithromycin.

Methods of treatment that involve frequent dosing often result in poor patient compliance. Moreover, for animals other than humans, treatment that involves multiple dosing can be difficult, labor intensive and costly, especially when the antibiotic is administered by injection.

### SUMMARY OF THE INVENTION

The invention relates to a method of treating a bacterial infection in an animal comprising administering to the animal a single dose of roxithromycin by injection.

In another embodiment, the invention relates to a method of treating a bacterial infection in a cat comprising administering to the cat a single dose of roxithromycin by injection or orally.

### BRIEF DESCRIPTION OF THE DRAWINGS

FIG. 1 is a graphical representation of the average plasma serum concentration of roxithromycin as a function of time when 5 cats were administered a single dose of roxithromycin by subcutaneous injection at a dose of 10 mg/kg as a formulation containing 200 mg/mL of roxithromycin in 10% propylene glycol in glycerol formal.

FIG. 2 is a graphical representation of the average plasma serum concentration of roxithromycin as a function of time when 5 cats were administered a single oral dose of roxithromycin at a dose of 20 mg/kg.

### DETAILED DESCRIPTION OF THE INVENTION

The invention relates to a method of treating a bacterial infection in an animal comprising administering to the animal a single dose of roxithromycin by injection.

In one embodiment, the animal is a mammal.

In another embodiment, the animal is selected from the group consisting of a cat, a dog, or cattle.

In another embodiment, the invention relates to a method of treating a bacterial infection in a cat comprising administering to the cat a single dose of roxithromycin by injection or orally.

The phrase “treating,” “treatment of,” and the like includes the amelioration or cessation of a specified condition, typically a bacterial infection.

The term “animal,” as used herein includes, but is not limited to, cattle, cow, horse, sheep, pig, ungulate, chimpanzee, monkey, baboon, chicken, turkey, mouse, rabbit, rat, guinea pig, dog, cat, and human.

The phrase “roxithromycin,” as used herein includes roxithromycin and pharmaceutically acceptable salts thereof. The phrase “pharmaceutically acceptable salt,” as used herein, is a salt formed from a basic nitrogen group of roxithromycin and an acid. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. In one embodiment, the method involves administering roxithromycin as the free base. In one embodiment, the method involves administering roxithromycin as a pharmaceutically acceptable salt of roxithromycin.

The phrase “single dose,” as used herein means a dose that is administered only once over a 28-day period. The dose may be administered in a single dosage form, such as a single injection or one capsule or tablet, or may be divided, e.g. constituted by more than one dosage form, such as by multiple capsules or tablets that are taken at or about the same time. The single dose is effective at treating a bacterial infection in an animal in need thereof.

The “single dose” used in the methods of the invention is formulated for immediate release and is not formulated for controlled or sustained release. For example, an orally administered roxithromycin single dose administered according to the methods of the invention is preferably in a form such that it releases roxithromycin to the gastrointestinal tract of the animal at a rate such that the total amount of roxithromycin is released from the dosage form in less than about 60 minutes. In one embodiment, the orally administered roxithromycin single dose administered according to the methods of the invention is in a form such that it releases roxithromycin to the gastrointestinal tract of the animal at a rate such that the total amount of roxithromycin is released from the dosage form in less than about 30 minutes. In one embodiment, the orally administered roxithromycin single dose administered according to the methods of the invention is in a form such that it releases roxithromycin to the gastrointestinal tract of the animal at a rate such that the total amount of roxithromycin is released from the dosage form in less than about 15 minutes.

In one embodiment, the dosage form used for single oral administration according to the methods of the invention meets the requirements for an immediate release dosage form as set forth in the FDA Guidelines, “Dissolution Testing of Immediate Release Solid Oral Dosage Forms, issued August, 1997, Section IV-A). Typically, at least 80% of the dosage form will dissolve in the first 60 minutes. In one embodiment, at least 80% of the dosage form will dissolve in the first 45 minutes. In one embodiment, at least 80% of the dosage form will dissolve in the first 30 minutes. In one embodiment, at least 80% of the dosage form will dissolve in the first 15 minutes. In one embodiment, the dissolution medium is 0.1 N HCl.

The phrase “elimination half life” or “T_{1/2},” as used herein, has the conventional meaning used in pharmacokinetics, i.e., the time taken for the maximum plasma concentration (Cₘₐₓ) of a drug to reduce by 50%.

The term “propylene glycol,” as that term is used herein, means CH₂(OH)CH₂CH₂(OH) or CH₂(OH)CH₂(OH)CH₃, i.e., 1,3-propylene glycol or 1,2-propylene glycol.

The term “glycerol formal,” as used herein means an organic solvent of formula C₄H₈O₃ that exists as a mixture of 5-hydroxy-1,3-dioxane and 4-hydroxymethyl-1,3-dioxolane in a ratio of about 60:40. Although the solvent glycerol formal consists of two chemical compounds, the two chemical compounds being in a specific ratio of about 60:40, it is typically considered a “solvent” rather than a mixture of compounds. This is because the 5-hydroxy-1,3-dioxane and 4-hydroxymethyl-1,3-dioxolane are in equilibrium with each other. Accordingly, the term glycerol formal (i.e., a mixture of 5-hydroxy-1,3-dioxane and 4-hydroxymethyl-1,3-dioxolane in a ratio of about 60:40), as used herein, is an organic solvent.

In one embodiment, the animal is a mammal.

In one embodiment, the animal is a dog.

In one embodiment, the animal is a cat.

In one embodiment, the animal is a cattle.

In one embodiment, the animal is a human.

The dose of roxithromycin is administered to the animal by injection.

In one embodiment, the dose of roxithromycin is administered to a mammal by injection.

In one embodiment, the dose of roxithromycin is administered to a cat by injection.

In one embodiment, the dose of roxithromycin is administered to a dog by injection.

In one embodiment, the dose of roxithromycin is administered to cattle by injection.

In one embodiment, the dose of roxithromycin is administered by subcutaneous injection.

In one embodiment, the dose of roxithromycin is administered by intramuscular injection.

In one embodiment, the dose of roxithromycin is administered intravenously.

For cats, however, the roxithromycin can also be administered orally. Accordingly, in one embodiment, the dose of roxithromycin is administered orally to a cat.

The amount of the roxithromycin that is effective at treating a bacterial infection can be determined by standard clinical techniques. The precise dose to be employed will also depend on the route of administration, the seriousness or severity of the bacterial infection, the susceptibility of the infecting organism to the roxithromycin, and the characteristics of the animal being treated and can be decided according to the judgment of a practitioner and/or each animal's circumstances.

The dose of roxithromycin, administered by injection is typically about 5 mg/kg or greater. In one embodiment, the dose of roxithromycin is about 10 mg/kg or greater. In one embodiment, the dose of roxithromycin is about 15 mg/kg or greater. In one embodiment, the dose of roxithromycin is about 20 mg/kg or greater. In one embodiment, the dose of roxithromycin ranges from about 5 mg/kg to about 50 mg/kg. In one embodiment, the dose of roxithromycin ranges from about 10 mg/kg to about 50 mg/kg. In one embodiment, the dose of roxithromycin ranges from about 20 mg/kg to about 50 mg/kg. In one embodiment, the dose of roxithromycin ranges from about 5 mg/kg to about 15 mg/kg. In one embodiment, the dose of roxithromycin ranges from about 5 mg/kg to about 20 mg/kg. In one embodiment, the dose of roxithromycin ranges from about 5 mg/kg to about 30 mg/kg. In one embodiment, the dose of roxithromycin ranges from about 10 mg/kg to about 40 mg/kg. In one embodiment, the dose of roxithromycin ranges from about 10 mg/kg to about 30 mg/kg. In one embodiment, the dose of roxithromycin ranges from about 10 mg/kg to about 20 mg/kg.

When treating a bacterial infection by orally administering a single dose of roxithromycin to a cat, the dose is typically about 2 to 3 times higher than if an injectable dosage form were administered. Typically, the injectable dose for a cat ranges from about 2 to about 25 mg/kg. In one embodiment, the injectable dose for a cat ranges from about 3 to about 20 mg/kg. In one embodiment, the injectable dose for a cat ranges from about 5 to about 15 mg/kg. In one embodiment, the injectable dose for a cat ranges from about 7 to about 12 mg/kg.

In one embodiment, the bacterial infections is caused by gram negative bacteria.

In one embodiment, the bacterial infections is caused by gram positive bacteria.

Representative bacterial infections that can be treated by the methods of the invention include, but are not limited to, bacterial infections caused by bacteria of the genus Clostridium, Pasteurella, Haemophilus, Fusobacterium, Moraxella, Bacteroides, Aeromonas, Escherichia, Enterobacter, Klebsiella, Listeria, Helicobacter, Legionella, Gardnerella, Salmonella, Shigella, Serratia, Ureaplasma, Chlamydia, Actinobacillus, Streptococcus, Edwardsiella, Staphylococcus, Enterococcus, Bordetella, Neisseria Proteus, Mycoplasma, Mannheimia, or Ureaplasma.

In one embodiment, the bacterial infection is caused by bacteria of the genus Clostridium, Streptococcus, Neisseria, Mycoplasma, Ureaplasma, Helicobacter, Listeria, Chlamydia, Legionella, Gardnerella, or Moraxella.

Representative bacterial infections that can be treated by the methods of the invention include, but are not limited to, bacterial infections caused by Pasteurella haemolytica, Clostridium perfinges, Pasteurella multocida, Pasteurella haemolytica, Haemophilus somnus, Actinobacillus pleuropneumoniae, Actinomyces pyogenes, Pseudomonas aeruginosa, Klebsiella pneumonia, Klebsiella oxytoca, Escherichiafaecalis, Escherichia coli, Staphylococcus aureaus, Staphylococcus intermedius, Enterococcus faecalis, Enterococcus faecium, Streptococcus pyogenes, Bacillus subtilis, Peptococcus indolicus, Mycoplasma bovis, Mycoplasma dispar, Mycoplasma hyopneumoniae, Mycoplasma hyorhinis, Mycoplasma gallisepticum, Mycoplasma mycoides, Mycoplasma ovipneumonia, Haemophilus infiuenzae, Klebsiella salmonella, Shigella, Proteus enterobacter, Enterobacter cloacae, Mannhemia haemolytica, Haemophilus somnus, Fusobacterium necrophorum, Bacterioides melaminogenicus, Proteus mirabillis, Streptococcus suis, Salmonella cholerasuis, Edwardsiella ictaluri, Aeromonas salmonicidia, Actinobaccilus pleuropneumoniae, and Bordetella bronchoseptica.

In one embodiment, the bacterial infection is caused by Streptococcus agalactiae, Streptococcus pneumoniae (Pneumococcus), Neisseria meningitides (Meningococcus), Listeria monocytogenes, Mycoplasma pneumoniae, Chlamydia trachomatis, Ureaplasma urealyticum, Legionella pneumophila, Helicobacter (Campylobacter)-Gardnerella vaginalis, Bordetella pertussis, Moraxella catarrhalis (Branhamella Catarrhalis), Haemophilus ducreyi, Group A beta-haemolytic Streptococci (Streptococcus pyogenes), Staphylococcus aureus, Haemophilus influenzae, or Staphylococcus epidermidis

In one embodiment, the bacterial infection is caused by Streptococcus agalactiae, Streptococcus pneumoniae (Pneumococcus), Neisseria meningitides (Meningococcus), Listeria monocytogenes, Mycoplasma pneumoniae, Chlamydia trachomatis, Ureaplasma urealyticum, Legionella pneumophila, Helicobacter (Campylobacter)-Gardnerella vaginalis, Bordetella pertussis, Moraxella catarrhalis (Branhamella catarrhalis), Haemophilus ducreyi.

Typically, to be effective, the minimum inhibitory concentration of the roxithromycin against a specific bacteria should be less than 10 μg/mL, preferably less than 5 μg/mL, more preferably less than 2 μg/mL, even more preferably less than 1 μg/mL, and most preferably less than 0.5 μg/mL. The activity of roxithromycin against a bacteria can be determined using standard dilution tests. For example, the minimum inhibitory concentrations can be determined using the disk diffusion susceptibility testing method described in Clinical Microbiology Procedures Handbook, volume 1, edited by Henry D. Isenberg, American Society for Microbiology, 1992, section 5.1 or the well known method of Bauer et al. “Antibiotic Susceptibility Testing by a Standardized Single Disc Method,” Amer. J. Clin. Pathol., 45, p. 493-496.

The method of the invention can be used to treat infections including, but not limited to, infections of the respiratory tract, eyes, ears, nose, throat, skin and skin structure, genito-urinary tract, and general systemic infections.

The elimination T_{1/2} for roxithromycin, when administered to a cat, was demonstrated to be about 73.5 hours. Therefore, the rate of clearance of roxithromycin from a cat, i.e., as measured by the T_{1/2}, is much slower than when roxithromycin is administered to, for example, a human. The unexpectedly slow rate of clearance (long T_{1/2}) allows bacterial infections in cats to be advantageously treated with a single administration of roxithromycin administered either orally or by injection. Treating a bacterial infection using a single dose of roxithromycin is simpler, more cost effective, and results in better patient compliance. Furthermore, single dose administration of roxithromycin is believed to reduce the risk of microbial strains emerging that are resistant to roxithromycin. Single dose administration of roxithromycin by injection is advantageous because administration by injection bypasses liver metabolism and the single administration precludes multiple visits to the veterinarian. Single dose administration orally, however, is less painful and can permit administration without requiring a visit to the veterinarian.

Typically, the roxithromycin is administered as a pharmaceutical composition, i.e., in combination with a suitable amount of a pharmaceutically acceptable excipient(s) so as to provide the form for proper administration to the animal., i.e., for administration by injection or, for cats, also by oral administration.

The pharmaceutical compositions are prepared by a method comprising admixing the roxithromycin and the pharmaceutically acceptable carrier or excipient. Admixing can be accomplished using methods well known for admixing a compound and a pharmaceutically acceptable carrier or excipient.

In one embodiment, the roxithromycin is formulated for subcutaneous injection, intramuscular injection, or intravenous administration. Compositions for subcutaneous injection, intramuscular injection, or intravenous administration can comprise sterile isotonic aqueous buffer. Where necessary, the compositions can also include a solubilizing agent. Non-aqueous compositions can also be used. Compositions for intravenous administration can optionally include a local anesthetic such as lidocaine to lessen pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the roxithromycin is to be administered by infusion, it can be dispensed, for example, with an infusion bottle containing sterile pharmaceutical grade solvent. Where the roxithromycin is administered by injection, an ampoule of sterile solvent, suitable for injection, can be provided so that the ingredients can be mixed prior to administration. In one embodiment, the solvent suitable for injection is an organic solvent.

In one embodiment, the roxithromycin is administered by injection as a solution in a mixture of propylene glycol and glycerol formal. In one embodiment, the roxithromycin is administered by injection as a solution in a mixture of about 10% propylene glycol in glycerol formal. In one embodiment, the roxithromycin is administered to cattle, a dog, or cat by injection as a solution in a mixture of propylene glycol and glycerol formal. In one embodiment, the roxithromycin is administered to cattle, a dog, or cat by injection as a solution in a mixture of about 10% propylene glycol in glycerol formal.

In one embodiment, the roxithromycin is formulated in accordance with routine procedures as a composition adapted for oral administration. Compositions for oral delivery can be in the form of tablets, lozenges, aqueous or oily suspensions, granules, powders, emulsions, capsules, syrups, or elixirs, for example. Tablet, pill, and capsule form are the preferred form for oral delivery. In one embodiment the roxithromycin is formulated as a composition adapted for oral administration, wherein release of the roxithromycin from the dosage form is delayed until the dosage form reaches the small intestines. For example, the roxithromycin can be formulated as an enteric coated tablet. Enteric coatings are coatings that dissolve at a pH range higher than about 5, typically between about pH 5-7. Illustrative enteric coatings include, but are not limited to cellulose acetate phthalate, hydroxypropylmethyl cellulose phthalate, polyvinyl acetate phthalate, carboxymethylethylcellulose, Eudragit L (poly(methacrylic acid, methylmethacrylate), 1:1 ratio), and Eudragit S (poly(methacrylic acid, methylmethacrylate, 1:2 ratio), and mixtures thereof. It is known that roxithromycin is acid labile and decomposes in acidic environments (See, J. Sun, et al., Impact of Pharmaceutical Dosage Forms on the Pharmacokinetics of Roxithromycin in Healthy Human Volunteers, J. Antimicrobial Chemotherapy, 55, 796-799 (2005); A. Hassanzadeh et al., Pediatric Erythromycins: A Comparison of the Properties of Erythromycin A and B 2′-Ethyl Succinates, J. Med. Chem., 49, 6334-6342 (2006); and M. Mordi et al., Acid Catalyzed Degradation of Clarithromycin and Erythromycin B: A Comparative Study Using NMR Spectroscopy, J. Med. Chem., 43, 467-474 (2000). Releasing the roxithromycin in the small intestines, rather than the stomach, avoids exposure of the roxithromycin to the acidic environment of the stomach and, thus, avoids its decomposition.

The pharmaceutical excipients can be liquids, such as water, organic solvents, and oils, including those of petroleum, animal, vegetable, or synthetic origin, such as peanut oil, soybean oil, mineral oil, sesame oil, and the like. Pharmaceutically acceptable excipients include, but are not limited to, binding agents, filling agents, lubricating agents, suspending agents, sweeteners, flavoring agents, preservatives, buffers, wetting agents, disintegrants, effervescent agents, coloring agents, pH buffering agents, and other excipients depending upon the route of administration and the dosage form desired. Such excipients are known in the art. Examples of suitable pharmaceutical excipients are described in Remington's Pharmaceutical Sciences 1447-1676 (Alfonso R. Gennaro ed., 19th ed. 1995), the contents of which are incorporated herein by reference.

Examples of filling agents are lactose monohydrate, lactose anhydrous, and various starches; examples of binding agents are various celluloses and cross-linked polyvinylpyrrolidone, microcrystalline cellulose, such as Avicel PH101 and Avicel PH102, microcrystalline cellulose, and silicified microcrystalline cellulose (ProSolv SMCC™).

Suitable lubricants, including agents that act on the flowability of the powder to be compressed, are colloidal silicon dioxide, such as Aerosil 200, talc, stearic acid, magnesium stearate, calcium stearate, and silica gel.

Examples of sweeteners are any natural or artificial sweetener, such as fructose, sucrose, xylitol, sodium saccharin, cyclamate, aspartame, and acsulfame. Examples of flavoring agents are Magnasweet (trademark of MAFCO); oil of wintergreen; bubble gum flavor; peppermint flavor; spearmint flavor; fruit flavors such as cherry, grape, orange; and tuna and other fish flavors and the like. Sweeteners and flavoring agents are particularly useful in orally administered dosage forms to provide a pharmaceutically palatable preparation.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel PH101 and Avicel PH102; lactose such as lactose monohydrate, lactose anhydrous, and Pharmatose DCL21; dibasic calcium phosphate such as Emcompress; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

In one embodiment, the pharmaceutically acceptable excipients are sterile when administered to an animal. Water, and in one embodiment physiological saline, can be used as excipient when the roxithromycin is administered intravenously. Saline solutions and aqueous dextrose and glycerol solutions can also be employed as liquid excipients, particularly for injectable solutions. In one embodiment, the liquid excipient is a non-aqueous solvent such as N-methyl-2-pyrollidone; a mixture of N-methyl-2-pyrollidone, polyethylene glycol, and propylene glycol; a mixture of propylene glycol and glycerol formal, or the solvents described in U.S. Pat. No. 5,082,863 to Apelian, the contents of which are expressly incorporated herein by reference.

Compositions for oral administration or administration by injection typically contain the roxithromycin in an amount ranging from about 1 percent to 80 percent by weight of the pharmaceutical compositions. In one embodiment, the compositions contain the roxithromycin in an amount ranging from about 5 percent to 75 percent by weight of the pharmaceutical compositions. In one embodiment, the compositions contain roxithromycin in an amount ranging from about 10 percent to 70 percent by weight of the pharmaceutical compositions. In one embodiment, the compositions contain roxithromycin in an amount ranging from about 10 percent to 55 percent by weight of the pharmaceutical compositions. In one embodiment, the compositions contain the roxithromycin in an amount ranging from about 15 percent to 65 percent by weight of the pharmaceutical compositions. In one embodiment, the compositions contain the roxithromycin in an amount ranging from about 20 percent to 55 percent by weight of the pharmaceutical compositions. In one embodiment, the compositions contain the roxithromycin in an amount ranging from about 1 percent to 10 percent by weight of the pharmaceutical compositions. In one embodiment, the compositions contain the roxithromycin in an amount ranging from about 2 percent to 7 percent by weight of the pharmaceutical compositions. In one embodiment, the compositions contain the roxithromycin in an amount ranging from about 1 percent to 25 percent by weight of the pharmaceutical compositions. In one embodiment, the compositions contain the roxithromycin in an amount ranging from about 5 percent to 25 percent by weight of the pharmaceutical compositions. In one embodiment, the compositions contain the roxithromycin in an amount ranging from about 15 percent to 25 percent by weight of the pharmaceutical compositions.

The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### EXAMPLES

### Example 1

### Single Dose Administration of Roxithromycin to Cats by Subcutaneous Injection

Five cats (mixed breed of various sizes, males and female, approximately 4 kg) were administered 10 mg/kg of a roxithromycin composition by subcutaneous injection between the shoulder blades. The roxithromycin composition was prepared by weighing 5.128 g of roxithromycin (purity 97.5%) into a 25 mL volumetric flask, adding 2.5 mL of propylene glycol, and filling to volume with stabilized glycerol formal.

Cats were kept in a stainless steel, suspended, wire bottom cage, at least 3′×3′, provided with a litter box in an environmentally controlled room (22±3° C., a relative humidity of 30-80%, a 12 hour light/dark schedule, and room ventilation of approximately 10-12 air changes per hour.) Tap water was available ad libitum and the cats were fed PMI Feline Lab Diet #5003 or other commercial product.

Blood samples were collected on Day-1 and approximately 1 mL of whole blood was collected at 1, 8, 24, 36, 48, 60, and 72 hr following dosing and twice-daily thereafter through study termination on day 10. The blood was separated to provide serum and the serum frozen and maintained for analysis. The cats were observed during dosing for any unusual reaction and clinical observations were made hourly for 8 hours following dosing. The dose site was monitored daily during the study.

The serum was analyzed by high pressure liquid chromatograph (“HPLC”) using the following procedure:

Transfer 500 μl of serum into a 15 ml centrifuge tube;

Add 10 μl of internal standard (“clarithromycin internal standard,” prepared as described below);

Add 2 mL acetonitrile;

Vortex for 20 seconds;

Sonicate for 5 minutes;

Centrifuge at 8250 rpm at 4° C. for 10 minutes;

Decant supernatant into a 4 mL vial;

Dry under nitrogen and heat and lyophilize for 15 minutes;

Reconstitute with 250 μl of 1:1 acetonitrile-buffer (25 mM sodium phosphate pH 7.2);

Vortex for 5 seconds;

Filter with 2 μm GHP Acrodisc 13 mm syringe filter;

Inject 10 μl onto an HPLC system equipped with an electrochemical detector using the following analytical parameters

HPLC Parameters::

| | |
|---|---|
| Column:: | Xbridge C8, 4.6 × 30 mm, 5 μm (Waters-186003194) |
| | column equipped with a Gemini C18, 4 × 3 mm |
| | (Phenomenex AJO-7597) guard column |
| Solvent: | A - 25 mM sodium phosphate pH 7.2 |
| | B - Acetonitrile |
| Initial condition: | 20% B 80% A, Flow: 1.5 mL/min |

Pump Schedule

| Time | % Solvent B | Flow (mL/min) |
|---|---|---|
| 3.00 | 20.0 | 1.5 |
| 15.00 | 38.0 | 1.5 |
| 15.50 | 60.0 | 1.5 |
| 20.00 | 60.0 | 1.5 |
| 20.50 | 20.0 | 1.5 |
| 40.00 | 20.0 | 1.5 |

The HPLC was equipped with a ESA CGIII CouloChem III electrochemical detector equipped with dual detectors (channel 1 and channel 2). The detector was operated using the following parameters:

Stop time: Same as pump
Rate of data acquisition: 5 data points/sec
Channel 1 operating parameters:

| Cell Potential: | 50 mV |
|---|---|
| Filter constant: | 5.0 sec |
| Full Scale Gain Range: | 100 nA |
| Signal Output Voltage: | 1.0 V |
| Baseline offset: | 0% |

Channel 2 operating parameters:

| | |
|---|---|
| Cell Potential: | 800 mV |
| Filter constant: | 5.0 sec |
| Full Scale Gain Range: | 100 nA |
| Signal Output Voltage: | 1.0 V |
| Baseline offset: | 0% |

The settings for the first and second channels were varied according to the following time schedule:

| Time | Event | Channel | Value |
|---|---|---|---|
| 0.01 | Auto zero | | |
| 19 | Set Cell potential (mV) | Both | 1000 |
| 20 | Set Cell potential (mV) | Both | −400 |
| 21 | Set Cell potential (mV) | 2 | 800 |
| 21 | Set Cell potential (mV) | 1 | 50 |

The guard column is changed and the column washed after every 50-100 injections. The column is washed according to the following sequence: 100% water, 10/90 water-methanol, 10/90 water-tetrahydrofuran (do not run tetrahydrofuran through the electrochemical detector), 100% acetonitrile.

The serum concentration of roxithromycin was then determined by comparing the area under the curve for the HPLC peak corresponding to roxithromycin to a standard curve of peak areas v. known concentrations of roxithromycin in serum. The standard curve was prepared using the following concentrations of roxithromycin 0.1, 1, 2, 4, 5, 10, 20, and 40 μg/mL. The solutions used to prepare the standard curve were prepared by the following procedure:

1. Prepare 100 mL of a roxithromycin solution at a concentration of 1 mg/ml in acetonitrile in a 100 mL volumetric flask. This is the roxithromycin stock solution.

2. Prepare 100 mL of a clarithromycin solution at a concentration of 1 mg/mL in acetonitrile in a 100 mL volumetric flask.

3. Prepare a solution clarithromycin at 100 μL/mL by transferring 100 μL of the solution prepared in step 2 into a 15 mL centrifuge tube, adding 900 μL of acetonitrile, and mixing well. This is the clarithromycin internal standard.

4. The following stock solutions are then prepared:
Stock solution A: 10 μL roxithromycin stock solution+990 μL acetonitrile=10 μL/mL.
Stock solution B: 100 μL roxithromycin stock solution+900 μL acetonitrile=100 μL/mL.
Stock solution C: 200 μL 1 roxithromycin stock solution+800 μL acetonitrile=200 μL/mL.
Stock solution D: 400 μL roxithromycin stock solution+600 μL acetonitrile=400 μL/mL.

5. The solutions used to prepare the standard curve are then obtained by combining
10 μL Stock solution A+990 μL serum=0.1 μL/mL.
10 μL Stock solution B+990 μL serum=1 μL/mL.
10 μL Stock solution C+990 μL serum=2 μL/mL.
10 μL Stock solution D+990 μL serum=4 μL/mL.
5 μL roxithromycin stock solution+995 μL serum=5 μL/mL.
10 μL roxithromycin stock solution+990 μL serum=10 μL/mL.
20 μL roxithromycin stock solution+980 μL serum=20 μL/mL.
40 μL roxithromycin stock solution+960 μL serum=40 μL/mL.

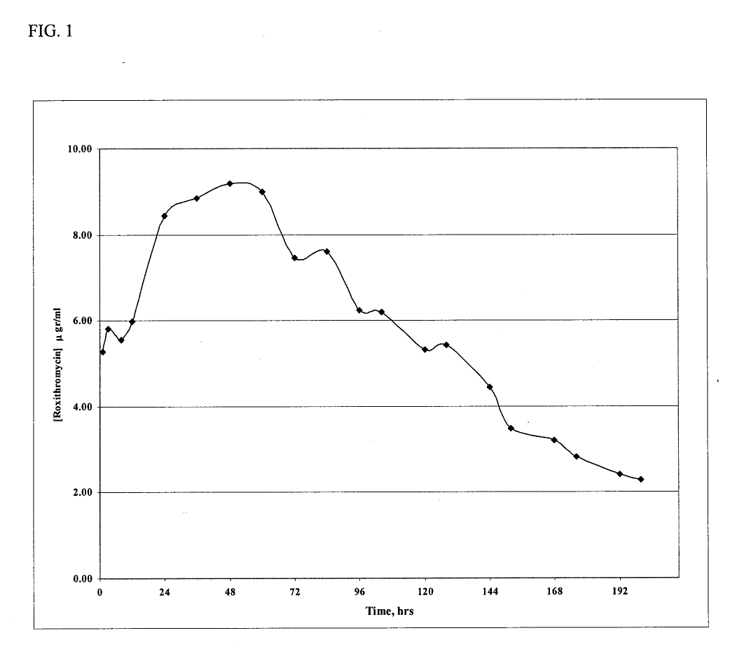

FIG. 1 provides a graphical representation of the average plasma serum concentration of roxithromycin as a function of time for the 5 cats that were administered a single dose of roxithromycin by subcutaneous injection at a dose of 10 mg/kg as a formulation containing 200 mg/mL of roxithromycin in 10% propylene glycol in glycerol formal. The data shows that for more than 190 hours the serum concentration for roxithromycin is sufficiently high that it exceeds the minimum inhibitory concentration (“MIC”) for several bacterial organisms. The table provided below shows the MIC for several organisms determined using the single disc method as described in Bauer et al. “Antibiotic Susceptibility Testing by a Standardized Single Disc Method,” Amer. J. Clin. Pathol., 45, p. 493-496.

| | Minimum Inhibitory Concentration (MIC) |
|---|---|
| Bacteria | μg/mL* |
| Staphylococcus aureus | 0.25 |
| Bacillus subtilis | 0.25 |
| Streptococcus pyogenes | 0.25 |
| Streptococcus pneumoniae | 0.25 |
| Clostridium perfringes | 2 |
| Enterococcus faecalis | 8 |
| Escherichia coli | 32 |
| Pasteurella multocida | 1 |

The results demonstrate that a single dose of roxithromycin, administered to a cat by subcutaneous injection at a dose of 10 mg/kg, provides a serum concentration of roxithromycin that is effective to treat bacterial infections including Staphylococcus aureus, Bacillus subtilis, Streptococcus pyogenes, Streptococcus pneumoniae, Clostridium perfringens, and Pasteurella multocida.

### Example 2

### Single Dose Oral Administration of Roxithromycin to Cats

Five cats (mixed breed of various sizes, males and female, approximately 4 kg) were administered orally a capsule containing 20 mg/kg of powdered roxithromycin. Blood samples (about 1 mL) were collected as a function of time. The blood was separated to provide serum and the serum frozen and maintained for analysis. The serum was then analyzed using HPLC as described above.

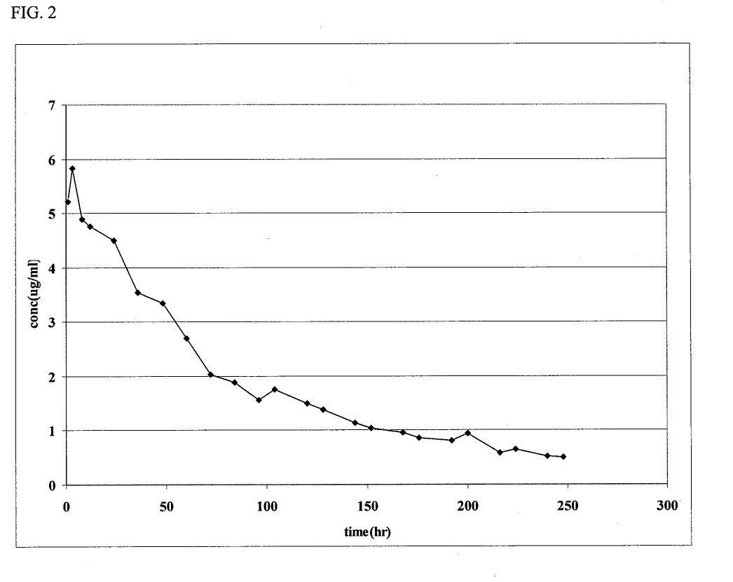

FIG. 2 provides a graphical representation of the average plasma serum concentration of roxithromycin as a function of time for the 5 cats that were administered a single oral dose of roxithromycin at a dose of 20 mg/kg. The data shows that for at least 150 hours, and even longer, the serum concentration for roxithromycin is sufficiently high that it exceeds the MIC for several bacterial organisms. The results demonstrate that a single dose of roxithromycin, orally administered to a cat at a dose of 20 mg/kg, provides a serum concentration of roxithromycin that is effective to treat bacterial infections including Staphylococcus aureus, Bacillus subtilis, Streptococcus pyogenes, Streptococcus pneumoniae, Clostridium perfringens, and Pasteurella multocida.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited, the entire disclosure of which are incorporated herein by reference.

What is claimed is:

1. A method of treating a bacterial infection in an animal comprising administering to the animal a single dose of roxithromycin by injection.

2. The method of claim 1, wherein the dose of roxithromycin is about 5 mg/kg or greater.

3. The method of claim 2, wherein the dose of roxithromycin ranges from about 5 mg/kg to about 50 mg/kg.

4. The method of claim 2, wherein the dose of roxithromycin is about 10 mg/kg or greater.

5. The method of claim 2, wherein the dose of roxithromycin is about 15 mg/kg or greater.

6. The method of claim 1, wherein the animal is a mammal.

7. The method of claim 1, wherein the animal is a cat.

8. The method of claim 1, wherein the animal is a dog.

9. The method of claim 1, wherein the animal is cattle.

10. The method of claim 1, wherein the administering is by subcutaneous injection.

11. The method of claim 1, wherein the bacterial infection is caused by bacteria of the genus Pasteurella, Haemophilus, Fusobacterium, Moraxella, Bacteroides, Aeromonas, Escherichia, Enterobacter, Klebsiella, Listeria, Helicobacter, Legionella, Gardnerella, Salmonella, Shigella, Serratia, Ureaplasma, Chlamydia, Actinobacillus, Streptococcus, Edwardsiella, Staphylococcus, Enterococcus, Bordetella, Neisseria Proteus, Mycoplasma, Mannheimia, or Ureaplasma.

12. The method of claim 1, wherein the bacterial infection is caused by Pasteurella haemolytica, Pasteurella multocida, Pasteurella haemolytica, Haemophilus somnus, Actinobacillus pleuropneumoniae, Actinomyces pyogenes, Pseudomonas aeruginosa, Klebsiella pneumonia, Klebsiella oxytoca, Escherichia faecalis, Escherichia coli, Staphylococcus aureaus, Staphylococcus intermedius, Enterococcus faecalis, Enterococcus faecium, Streptococcus pyogenes, Bacillus subtilis, Peptococcus indolicus, Mycoplasma bovis, Mycoplasma dispar, Mycoplasma hyopneumoniae, Mycoplasma hyorhinis, Mycoplasma gallisepticum, Mycoplasma mycoides, Mycoplasma ovipneumonia, Haemophilus influenzae, Klebsiella salmonella, Shigella, Proteus enterobacter, Enterobacter cloacae, Mannhemia haemolytica, Haemophilus somnus, Fusobacterium necrophorum, Bacterioides melaminogenicus, Proteus mirabillis, Streptococcus suis, Salmonella cholerasuis, Edwardsiella ictaluri, Aeromonas salmonicidia, Actinobaccilus pleuropneumoniae, and Bordetella bronchoseptica.

13. The method of claim 1, wherein the bacterial infection is an infection of the respiratory tract, eyes, ears, nose, throat, skin and skin structure, genito-urinary tract, or general systemic infection.

14. The method of claim 1, wherein the roxithromycin is administered as a solution in mixture of propylene glycol and glycerol formal.

15. The method of claim 14, wherein the solution is a solution of about 10% propylene glycol in glycerol formal.

16. The method of claim 15, wherein the concentration of roxithromycin in the 10% propylene glycol in glycerol formal is about 200 mg/mL.

17. The method of claim 16, wherein the roxithromycin is administered at a dose ranging from about 5 mg/kg to about 50 mg/kg.

18. The method of claim 17, wherein the animal is a cat, a dog, or cattle.

19. The method of claim 18, wherein the animal is a cat and the roxithromycin is administered at a dose of about 10 mg/kg.

20. The method of claim 17, wherein the animal is a dog.

21. A method of treating a bacterial infection in a cat comprising orally administering to the cat a single dose of roxithromycin.

22. The method of claim 21, wherein the dose of roxithromycin is about 5 mg/kg or greater.

23. The method of claim 22, wherein the dose of roxithromycin ranges from about 5 mg/kg to about 50 mg/kg.

24. The method of claim 23, wherein the dose of roxithromycin is about 15 mg/kg or greater.

25. The method of claim 24, wherein the dose of roxithromycin is about 20 mg/kg or greater.

26. The method of claim 21, wherein the bacterial infection is caused by bacteria of the genus Pasteurella, Haemophilus, Fusobacterium, Moraxella, Bacteroides, Aeromonas, Escherichia, Enterobacter, Klebsiella, Listeria, Helicobacter, Legionella, Gardnerella, Salmonella, Shigella, Serratia, Ureaplasma, Chlamydia, Actinobacillus, Streptococcus, Edwardsiella, Staphylococcus, Enterococcus, Bordetella, Neisseria Proteus, Mycoplasma, Mannheimia, or Ureaplasma.

27. The method of claim 21, wherein the bacterial infection is caused by Pasteurella haemolytica, Pasteurella multocida, Pasteurella haemolytica, Haemophilus somnus, Actinobacillus pleuropneumoniae, Actinomyces pyogenes, Pseudomonas aeruginosa, Klebsiella pneumonia, Klebsiella oxytoca, Escherichiafaecalis, Escherichia coli, Staphylococcus aureaus, Staphylococcus intermedius, Enterococcus faecalis, Enterococcus faecium, Streptococcus pyogenes, Bacillus subtilis, Peptococcus indolicus, Mycoplasma bovis, Mycoplasma dispar, Mycoplasma hyopneumoniae, Mycoplasma hyorhinis, Mycoplasma gallisepticum, Mycoplasma mycoides, Mycoplasma ovipneumonia, Haemophilus influenzae, Klebsiella salmonella, Shigella, Proteus enterobacter, Enterobacter cloacae, Mannhemia haemolytica, Haemophilus somnus, Fusobacterium necrophorum, Bacterioides melaminogenicus, Proteus mirabillis, Streptococcus suis, Salmonella cholerasuis, Edwardsiella ictaluri, Aeromonas salmonicidia, Actinobaccilus pleuropneumoniae, and Bordetella bronchoseptica.

28. The method of claim 21, wherein the bacterial infection is an infection of the respiratory tract, eyes, ears, nose, throat, skin and skin structure, genito-urinary tract, or general systemic infection.
